(19) United States
(12) Patent Application Publication
(10) Pub. No.: US 2008/0020412 A1
(43) Pub. Date: Jan. 24, 2008

(54) Aurora expression constructs

(76) Inventors: Michael J. Romanowski, Pflugerville, TX (US); L Michael Randal, Portolla Valley, CA (US)

Correspondence Address: HELLER EHRMAN LLP, 275 MIDDLEFIELD ROAD, MENLO PARK, CA, 94025-3506, US

(21) Appl. No.: 11/649,725

(22) Filed: Jan. 3, 2007

Related U.S. Application Data
(60) Provisional application No. 60/756,093, filed on Jan. 3, 2006.

(51) Int. Cl. C12N 9/12; C07H 21/00; C07K 14/00; C12Q 1/48; C12N 1/21; C12N 15/63

(52) U.S. Cl. 435/15; 435/194; 435/252.3; 435/252.33; 435/320.1; 530/350; 536/23.1; 536/23.2

(57) ABSTRACT

The present invention is directed to Aurora constructs that are useful for structural and functional studies of Aurora. Specifically, engineered Aurora enzymes are provided, along with polynucleotides encoding said enzymes, vectors comprising said polynucleotides, and host cells comprising the vectors. In addition, a process of using the engineered Aurora enzymes to identify Aurora modulators is provided.

This application claims the benefit of U.S. Provisional Application No. 60/756,093, filed Jan. 3, 2006, which is incorporated herein in its entirety.

### BACKGROUND

The Aurora enzymes are a family of serine-threonine kinases having roles in cell division that are composed of a variable N-terminal domain, a conserved catalytic domain and a very short C-terminal domain. Because of the roles of the Aurora enzymes in cell division, they are considered to be important therapeutic targets for cancer [Brown, J. R. et al., BMC Evolutionary Biology 4:39 (2004)].

Aurora overexpression and/or amplification has been observed in cervical cancer, ovarian cancer, and neuroblastoma cell lines [Warner, S. L. et al., Molecular Cancer Therapeutics 2:589-95 (2003)]. Furthermore, Aurora overexpression and/or amplification has been observed also in primary clinical isolates of bladder, breast, colorectal, gastric, and pancreatic cancers. Additionally, the expression level of Aurora has been associated with aggressiveness of certain types of cancers.

Despite some progress in characterization of Aurora enzymes, active Aurora enzymes can be toxic in the environment of certain expression systems used to produce them. In these systems, Aurora enzymes are subject to negative selection pressure, resulting in irreproducibility in enzyme isolation. For example, Aurora enzymes isolated from bacteria often contain mutations as compared with the expected products of the coding sequences used to express them that either inactivate the enzymes or greatly attenuate their activities. In addition, many Aurora constructs if they can be isolated at all, are unstable. Thus, there is a need for stable Aurora enzymes and expression constructs that will allow facile production of Aurora enzymes—particularly of active Aurora enzymes—to aid in the discovery of novel cancer therapeutics.

### SUMMARY

The present invention is directed to Aurora constructs that are useful for structural and functional studies of Aurora. Specifically, engineered Aurora enzymes are provided, along with polynucleotides encoding said enzymes, vectors comprising said polynucleotides, and host cells comprising the vectors. In addition, a process of using the engineered Aurora enzymes to identify Aurora modulators is provided.

### DESCRIPTION OF THE FIGURES

FIG. 1 shows an alignment of the catalytic domains for wild type mouse Aurora-B, wild type human Aurora-B, wild type human Aurora-A, and wild type mouse Aurora-A. The sequences are provided respectively as SEQ ID NO:1, SEQ ID NO:2, SEQ ID NO:3 and SEQ ID NO:4 in the Sequence Listing. All numbering in the figure is with respect to the full length wild type sequence for each protein. Residues varying among the four proteins are shown in bold. Residues underlined and in grey correspond to positions where mutations are present in at least one of the engineered Aurora-B constructs described herein to a less hydrophobic amino acid residue. The subset of residues in grey that are in italics are predicted to be spatially contiguous. Residues underlined and in black italics indicate a site of proteolysis.

FIG. 2 shows an alignment of the catalytic domain sequences of Aurora-B enzymes from human, mouse, and rat, and a fragment of the catalytic domain from pig. The sequences are provided respectively as SEQ ID NO:2, SEQ ID NO:1, SEQ ID NO: 5 and SEQ ID NO:6 in the Sequence Listing. All numbering in the figure is with respect to the full length wild type sequence for each protein, except for the pig sequence, which is numbered according to the fragment shown. Residues varying among the four proteins are shown in bold, and positions of mutations are underlined. Residues in grey italics are predicted to be spatially contiguous. Residues underlined and in black italics indicate a site of proteolysis.

FIG. 3 shows Aurora-A expression and purification. Panel (a): Mouse Aurora A residues 116-381 (human residues 125-391). M: Mark12 molecular-mass standard (Invitrogen); 1: induced cell lysate (insoluble and soluble protein); 2: supernatant; 3: supernatant after loading onto glutathione Sepharose; 4 & 5: top of protein peak eluted with 15 mM reduced glutathione; 6 & 7: total eluted protein; 8: total eluted protein digested for 2 hrs at 4° C. with PreScission protease; 9: GST; 10: same as lanes 6 & 7; 11: total eluted protein digested with PreScission protease overnight at 4° C.; 12: protein sample after removal of GST on glutathione Sepharose; 13: GST; 14: same as sample in lane 12; 15: protein after subtraction of GroEL on Q Sepharose; 16: GST. Panel (b): Mouse Aurora A residues 98-395 (human residues 107-403). M: Mark12 molecular-mass standard. 1: induced cell lysate (insoluble and soluble protein); 2: supernatant; 3: supernatant after loading onto glutathione Sepharose; 4: top of protein peak eluted with 15 mM reduced glutathione; 5: total eluted protein; 6: GST; 7: same as sample in lane 5; 8: total eluted protein digested with PreScission protease overnight at 4° C.; 9: protein sample after removal of GST on glutathione Sepharose; 10: GST; 11: same sample as in lane 9; 12: protein after subtraction of GroEL on Q Sepharose; 13: GST.

FIG. 4 shows Aurora-A activity assays. Panel (a): enzyme titration; panel (b): ATP titrations. Endpoint assay with ΔF representing an increase in fluorescence signal between sample and negative control during a 1-hr period. The maximum value for ΔF within a linear range is approximately 15,000 units. The Kₘ values and confidence intervals were determined by fitting the ATP titration curve in GraphPad Prism. The different mouse Aurora-A variants are indicated as follows: insect cell-expressed 98-395 [human residues 107-403] (●) with Kₘ=13.6±3.9 μM and Vₘₐₓ/[enzyme]=590 1/(hr·nM); E. coli-produced 98-395 (∘) with Kₘ=10.5±3.5 μM and Vₘₐₓ/[enzyme]=1035 1/(hr nM); E. coli-produced 116-381 [human residues 125-391] (□) with Kₘ=65.3±16.1 μM and Vₘₐₓ/[enzyme]=280 1/(hr nM); and the Gly133→Val variant, clone A4-20-1-23-R5 (Δ), which has little activity (Kₘ or Vₘₐₓ values not determined).

FIG. 5 shows the kinase activity in a homogeneous time resolved fluorescence (HTRP) assay at increasing concentrations of the following engineered Aurora-B constructs: Aurora B 8λ.VK→LQ, Aurora B 7X, and Aurora B 8X.VK→LR. Df is a normalized emission ratio, multiplied by 10⁴, as described further in Example 5.

### DETAILED DESCRIPTION OF THE INVENTION

Engineered Aurora Enzymes

In one aspect, the invention is directed to engineered Aurora-B enzymes. Engineered Aurora-B enzymes are proteins that have Aurora-B enzymatic activity as defined herein, and that have at least one substitution mutation in accordance with the present invention with respect to the corresponding wild type Aurora-B sequence. Preferably, the engineered Aurora-B enzymes of the invention have a sequence derived from a vertebrate Aurora-B. It is a finding of the invention that certain Aurora-B enzymes have a proteolysis site located between the position equivalent to V286 of full length wild type human Aurora-B and the position equivalent to K287 of full length wild type human Aurora-B. A mutation of at least one of the two residues immediately surrounding this proteolysis site aids in making active Aurora-B enzyme preparations. Without being bound by a particular theory, the mutation appears to contribute to the enzymatic stability and/or activity.

The engineered Aurora-B enzymes of the invention can be full length or enzymatically active fragments thereof. Unless otherwise indicated, for the engineered Aurora-B enzymes described herein, numbering is with respect to the Swiss Prot reference sequence for full length human Aurora-B (Accession No. Q96GD4), the sequence of which is provided as SEQ ID NO:7.

| | |
|---|---|
| MAQKENSYPW PYGRQTAPSG LSTLPQRVLR KEPVTPSALV LMSRSNVQPT | SEQ ID NO:7 |
| AAPGQKVMEN SSGTPDILTR HFTIDDFEIG RPLGKGKFGN VYLAREKKSH | |
| FIVALKVLFK SQIEKEGVEH QLRREIEIQA HLHHPNILRL YNYFYDRRRI | |
| YLILEYAPRG ELYKELQKSC TFDEQRTATI MEELADALMY CHGKKVIHRD | |
| IKPENLLLGL KGELKIADFG WSVHAPSLRR KTMCGTLDYL PPEMIEGRMH | |
| NEKVDLWCIG VLCYELLVGN PPFESASHNE TYRRIVKVDL KFPASVPTGA | |
| QDLISKLLRH NPSERLPLAQ VSAHPWVRAN SRRVLPPSAL QSVA | |

There are a number of naturally occurring variations of human Aurora-B, any of which may be used in the current invention. Known substitution variations include R14D, Q15K, E161M, Q167H, S169T, P226T, M249I, H250D, and T298M. Known insertion variations include an arginine inserted between residues corresponding to residue 70 and residue 71 of SEQ ID NO:7; a VRR inserted between residues corresponding to residue 179 and residue 180 of SEQ ID NO:7; and a RAV inserted between residues corresponding to residue 180 and residue 181 of SEQ ID NO:7. Known deletion variations include deletion of a residue corresponding to residue P271 of SEQ ID NO:7.

In one embodiment, the engineered Aurora-B enzyme comprises a substitution mutation of at least one residue in a position corresponding to V286 of SEQ ID NO:7 or a position corresponding to K287 of SEQ ID NO:7. In another embodiment, the engineered Aurora-B polypeptide comprises a substitution mutation in a position corresponding to V286. In another embodiment, the engineered Aurora-B enzyme comprises a substitution mutation in a position corresponding to K287. In another embodiment, the engineered Aurora-B enzyme comprises a substitution mutation in a position corresponding to V286 and comprises a substitution mutation in a position corresponding to K287.

The substitution mutation in the position corresponding to V286 may be to a variety of different amino acids. In one embodiment, the engineered Aurora-B enzyme comprises a substitution mutation in a position corresponding to V286, wherein the substitution mutation is to an amino acid isosteric with Leu. As defined herein, an amino acid isosteric with Leu may be Leu itself. The amino acid isosteric with Leu may be an unnatural or a natural amino acid.

Thus in another embodiment, the engineered Aurora-B enzyme comprises a V286 substitution mutation to an amino acid isosteric with Leu, wherein the isosteric amino acid is an unnatural amino acid. In yet another embodiment, the engineered Aurora-B enzyme comprises a V286 substitution mutation to oxonorvaline, leucic acid, norleucine, 2(O-methylthreonine), 3(isoleucine), 4(allo-O-methylthreonine) or 5(allo-leucine).

Alternatively, in a different embodiment, the engineered Aurora-B enzyme comprises a substitution mutation in a position corresponding to V286, wherein the mutation is to an amino acid isosteric with Leu, and wherein the isosteric amino acid is a natural amino acid. In another embodiment, the engineered Aurora-B enzyme comprises a V286 substitution mutation to Ser, Leu, Ile, Thr or Ala. In another embodiment, the engineered Aurora-B enzyme comprises a V286 substitution mutation to Ser or Leu.

The engineered Aurora-B enzyme may comprise, in another embodiment, a substitution mutation in a position equivalent to residue V286 of SEQ ID NO:7, wherein the substitution mutation is to a polar amino acid other than Ser. For example, the engineered Aurora-B enzyme may comprise a V286 substitution mutation to Gln or Asn.

In another embodiment, the engineered Aurora-B enzyme comprises a substitution mutation in a position corresponding to V286, wherein the substitution mutation is to a charged amino acid residue. In another embodiment, the engineered Aurora-B enzyme comprises a substitution mutation in a position corresponding to V286, wherein the substitution mutation is to Asp or Glu. In another embodiment, the engineered Aurora-B enzyme comprises a substitution mutation in a position corresponding to V286, wherein the substitution mutation is to Lys, Arg or His.

In another embodiment, the engineered Aurora-B enzyme comprising the amino acid substitution mutation in a position corresponding to V286 further comprises a substitution mutation in a position corresponding to K287. The amino acid substitution mutation in the position corresponding to K287 may be to a variety of amino acids.

In one embodiment the engineered Aurora-B enzyme comprises a substitution mutation in a position corresponding to V286 and a substitution mutation in a position corresponding to K287, wherein the substitution mutation in the position corresponding to K287 is to a polar amino acid residue. In another embodiment, the substitution mutation in the position corresponding to K287 is to Gln or Asn. In another embodiment, the substitution mutation in the position corresponding to K287 is to Gln. In another embodiment, the substitution mutation in the position corresponding to K287 is to a charged amino acid residue. In another embodiment, the substitution mutation in the position corresponding to position K287 is to Arg.

Certain engineered Aurora-B enzymes of the invention further comprise a substitution mutation of at least one hydrophobic residue predicted to be on the protein surface to a less hydrophobic amino acid. In one embodiment, the surface residue is a leucine. In another embodiment the engineered Aurora-B enzyme further comprises at least one substitution mutation in a position corresponding to residue L210 or L228 of SEQ ID NO:7. In another embodiment, the engineered Aurora-B further comprises a substitution mutation in a position corresponding to residue L210 of SEQ ID NO:7. In another embodiment, the engineered Aurora-B further comprises a substitution mutation in a position corresponding to residue L228 of SEQ ID NO:7. In another embodiment, the engineered Aurora-B enzyme further comprises a substitution mutation in a position corresponding to L210 of SEQ ID NO: 7 and a substitution mutation in a position corresponding to L228 of SEQ ID NO:7.

The leucine residues predicted to be on the protein surface may be mutated to a variety of amino acid residues less hydrophobic than leucine. In one embodiment, the leucine residue predicted to be on the protein surface is mutated to Lys, Arg, His, Glu or Asp. In another embodiment, the leucine residue predicted to be on the protein surface is mutated to Gly, Asn, Gln, Cys, Ser or Thr. In another embodiment, the leucine residue predicted to be on the protein surface is mutated to Ser or Thr.

In yet other embodiments, the engineered Aurora-B enzymes may comprise at least one substitution mutation of at least one proline residue predicted to be on the protein surface. In one embodiment the engineered Aurora-B enzyme further comprises at least one substitution mutation in a position corresponding to residue P297 or P317 of SEQ ID NO:7. In another embodiment, the engineered Aurora-B further comprises a substitution mutation in a position corresponding to residue P297 of SEQ ID NO:7. In another embodiment, the engineered Aurora-B further comprises a substitution mutation in a position corresponding to residue P317 of SEQ ID NO:7. In another embodiment, the engineered Aurora-B enzyme further comprises a substitution mutation in a position corresponding to P297 of SEQ ID NO:7 and a substitution mutation in a position corresponding to P317 of SEQ ID NO:7.

The proline residues predicted to be on the protein surface may be mutated to a variety of amino acid residues less hydrophobic than proline. In one embodiment, the proline residue predicted to be on the protein surface is mutated to Lys, Arg, Asp or Glu. In another embodiment, the proline residue predicted to be on the protein surface is mutated to Gly, Asn, Gln, Cys, Ser or Thr. In another embodiment, the proline residue predicted to be on the protein surface is mutated to Ser or Thr.

Engineered Aurora-B enzymes of the invention may further comprise one or more of the naturally occurring variations of the human protein, with numbering corresponding to SEQ ID NO:7. In one embodiment, the engineered Aurora-B enzyme comprises the following variations: P226T, M2491, H250D, and a deletion of P271. In another embodiment, the engineered Aurora-B enzyme comprises the following variations: E161M, Q167H, S169T, and an insertion of VRR between residue 179 and residue 180 of SEQ ID NO:7. In another embodiment, the engineered Aurora-B enzyme comprises the following variations: R14D, Q15K, E161M, 1180V, and an insertion of RAV between residue 180 and residue 181 of SEQ ID NO:7. In yet another embodiment, the engineered Aurora-B enzyme comprises the following variations: T298M, and an insertion of an arginine between residues corresponding to residue 70 and residue 71 of SEQ ID NO:7.

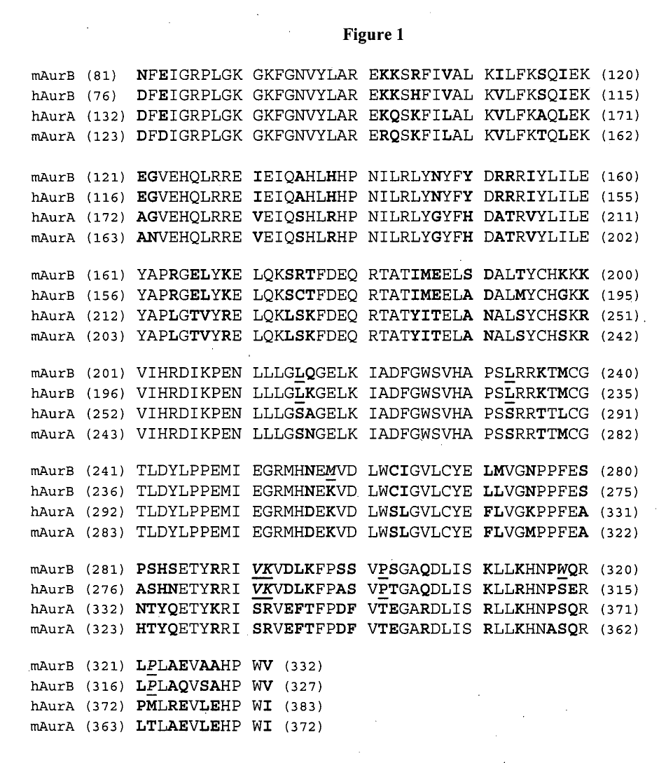

In addition to engineered Aurora-B enzymes having sequences derived from human, the engineered Aurora-B enzymes of the present invention also encompass enzymes having sequences derived from other species. In one embodiment, the engineered Aurora-B enzyme has a sequence derived from a mammalian Aurora-B. In another embodiment, the engineered Aurora-B enzyme has a sequence derived from a mouse Aurora-B. In another embodiment, the engineered Aurora-B enzyme has a sequence derived from a rat Aurora-B. In another embodiment, the engineered Aurora-B enzyme has a sequence derived from a pig Aurora-B. An alignment of the catalytic domain sequences of human, mouse, rat, and pig Aurora-B enzymes is provided in FIG. 1 herein as an illustration.

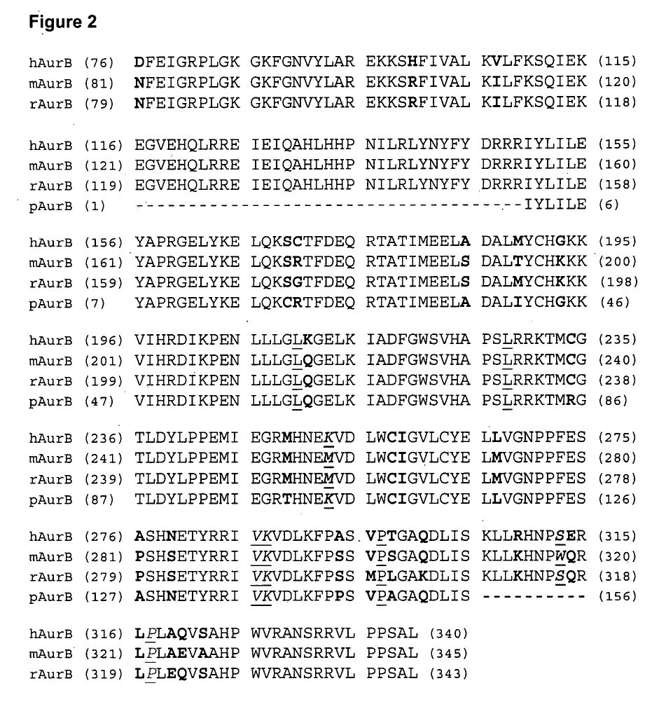

For engineered Aurora-B enzymes derived from certain species other than human, further substitution mutations can also be made. For example in the position equivalent to K253 of the human sequence (SEQ ID NO:7), mouse Aurora-B has a methionine (i.e. M258 of the mouse sequence). It is another finding of the invention that mutation of residue M258 of the mouse sequence contributes to active Aurora enzyme preparations. Thus in another embodiment, the invention is directed to an engineered Aurora-B enzyme derived from a mouse Aurora-B sequence, wherein the enzyme comprises a substitution mutation of a residue in a position equivalent to M258. To illustrate, a numbered alignment of mouse Aurora-B, human Aurora-B, human Aurora-A and mouse Aurora-B is provided in FIG. 2.

It is predicted herein that M258 is on the surface of the protein. As methionine can be hydrophobic, it is generally preferred that the substitution mutation be to an amino acid residue that is less hydrophobic than Met so that the solubility of the resulting protein may be increased. Thus, in another embodiment, the mutation in the position equivalent to M258 of the mouse sequence is to a charged amino acid residue. In another embodiment, the mutation in the position equivalent to M258 of the mouse sequence is to Asp or Glu. In another embodiment, the mutation in the position equivalent to M258 is to Lys, Arg, or His. In another embodiment, the mutation in the position equivalent to M258 is to Lys. In another embodiment, the mutation in the position equivalent to M258 is to Gly, Asn, Gln, Cys, Ser or Thr. In another embodiment, the mutation in the position equivalent to M258 is to Asn or Gln.

It is further predicted herein that in mouse Aurora-B enzymes, M258 is likely to be present in a patch of spatially contiguous hydrophobic surface residues. In particular, the patch is also likely to contain residues in a position equivalent to W318 of mouse Aurora-B, which corresponds to residue S313 of the human sequence (SEQ ID NO:7). In another embodiment, the engineered Aurora-B enzyme comprising a substitution mutation of a residue in a position equivalent to M258 of mouse Aurora-B further comprises a substitution mutation in a position equivalent to W318 of mouse Aurora-B. In another embodiment the substitution mutation in the position equivalent to W318 of mouse Aurora-B is to Gly, Asn, Gln, Cys, Ser or Thr. In another embodiment, the substitution mutation in the position equivalent to W318 of mouse Aurora-B is to Ser or Thr.

In another embodiment, the engineered Aurora-B enzyme comprises a sequence at least 85% identical to residues 81-332 of SEQ ID NO:7. In another embodiment, the engineered Aurora-B enzyme comprises a sequence at least 90% identical to residues 81-332 of SEQ ID NO:7. In another embodiment, the engineered Aurora-B enzyme comprises a sequence at least 95% identical to residues 81-332 of SEQ ID NO:7.

Percent identity of a protein sequence to a reference protein sequence, is determined by alignment of the protein sequence to the reference protein sequence using the GAP program [Huang, X., Computer Applications in the Biosciences 10:227-235 (1994)] in the Wisconsin Genetics Software Package Release 10.0, with a BLOSUM62 comparison matrix [Henikoff, S. & Henikoff, J. G., Proc Natl Acad Sci USA 89:10915-10919 (1992)] and default parameters for gap openings and gap extensions. The alignment is calculated over the entire length of the reference sequence, with no penalty for gaps outside the alignment to the reference sequence. The program GAP is an implementation of the Needleman-Wunsch algorithm [Needleman & Wunsch, J. Mol. Biol., 48:443-453 (1970)]. Positions of equivalence of a protein sequence to a reference protein sequence can also be determined by aligning the protein sequence to the reference sequence using the GAP program with the comparison matrix and default parameters described herein.

Engineered Aurora-B enzymes of the present invention preferably further comprise a solubilizing tag. Depending on the nature of the solubilizing tag, the tag may be fused N-terminal to or C-terminal to the Aurora enzyme. Numerous tags aiding in protein purification, expression, secretion and detection are reviewed by Stevens, R. C. [Structure 8:R177-R185 (2000)]. The solubilizing tags used herein may or may not possess an affinity moiety. Examples of solubilizing tags possessing an affinity moiety are GST and MBP. If the solubilizing tag does not have an affinity moiety, the Aurora enzyme may be expressed as a fusion containing the solubilizing tag lacking the affinity moiety as well as a separate affinity moiety. For example a His₆ affinity moiety may be expressed along with a NusA solubilizing tag to aid in the purification of the resulting Aurora fusion protein.

In yet another aspect, the invention is directed to a polypeptide comprising a sequence selected from the group consisting of SEQ ID NO:8, SEQ ID NO:9, and SEQ ID NO:10. In another embodiment, the polypeptide comprising a sequence selected from the group consisting of SEQ ID NO:8, SEQ ID NO:9, and SEQ ID NO:10 further comprises a solubilizing tag.

In another embodiment, the engineered Aurora enzymes of the invention further comprise a protease cleavage site situated between the solubilizing tag and the Aurora enzyme sequence. The protease cleavage site allows removal of the solubilizing tag. Non-limiting examples of specifically cleaving proteases that may be used to isolate the Aurora protein from a solubilizing tag include thrombin, tobacco etch virus (TEV) protease, Genenase I, Enterokinase, Granzyme B, turnip mosaic virus protease NIa, Factor Xa, and PreScission™ protease.

“Aurora-B enzymatic activity” as used herein is defined as kinase activity detectable above a negative control not containing Aurora in the Kinase Assay Protocol on page 2 of the Certificate of Analysis of Upstate Catalog #14-489, Lot # 24403, using an enhanced chemiluminescence detection method. The protocol is incorporated herein by reference.

Expression of Aurora Polypeptides in Bacterial Expression Systems

In another aspect, the invention is directed to a polynucleotide encoding an engineered Aurora enzyme. In another aspect, the invention is directed to an expression vector comprising the polynucleotide encoding the engineered Aurora enzyme. In certain embodiments of the expression vectors for engineered Aurora enzymes, the expression vectors coexpress a phosphatase.

In another aspect, the invention is directed to a bacterial host cell comprising the expression vector comprising the polynucleotide encoding the engineered Aurora enzyme.

In another embodiment, the Aurora encoding sequence encodes an Aurora-B enzyme having a GST tag fused at its N-terminus. In another embodiment, the Aurora enzyme encoding sequence encodes SEQ ID NO:8, SEQ ID NO:9, or SEQ ID NO:10 having a GST tag fused at its N-terminus.

In another embodiment, the polynucleotide comprises a bacterially functional operon, which is operably linked to
a) a first ribosomal binding site operably linked to an engineered Aurora encoding sequence, and
b) a second ribosomal binding site operably linked to a phosphatase encoding sequence.

In certain polynucleotides of the invention, the Aurora enzyme and the phosphatase are each located downstream of a respective ribosomal binding site. In prokaryotes, ribosomal binding sites are also known as Shine-Dalgarno (SD) sequences (Shine, J. & Dalgarno, L., Proc. Nat. Acad. Sci. USA 71:1342-1346 (1974)). Ribosomal binding sites in general have a good degree of complementarity to certain regions of the ribosomal RNA (rRNA) in the organism from which both are derived. Ribosomal binding site sequences are available for numerous prokaryotic organisms; see for example, Ma, J. et al., J. Bacteriol. 184:5733-5745 (2002). The consensus ribosomal binding site in E. coli has the purine-rich sequence 5′-AGGAGG-3′. For less well-characterized prokaryotes, there are programs available, for example, ORPHEUS, (Frishman, D. et al., Gene 234:257-65) that predict ribosomal binding sites.

For optimal translation, the ribosomal binding site is preferably placed at the optimal distance from the start codon. In general, for prokaryotes the optimal distance between the 3′-end of the ribosomal binding site and the start codon is 4 to 9 nucleotides. In E. coli, the optimal spacing between the ribosomal binding site and the start codon was determined to be five nucleotides [Chen, H. et al., Nucleic Acids Res. 22:4953-7 (1994)]. Furthermore, for E. coli, statistical analysis has been performed for base preferences within certain distances of the start codon, [see, for example, Barrick, D. et al., Nucleic Acids Res. 22:1287-95 (1994)].

In another embodiment, the Aurora encoding sequence and/or the phosphatase encoding sequence may be optimized for expression in a particular organism. Different organisms can have distinct preferences of codon usage. The codon usage preferences of hundreds of species have been catalogued [Nakamura, Y., et al., Nucl. Acids Res. 28: 292 (2000)]. For example, E. coli codon usage preferences have been particularly well characterized [Ikemura, T., J. Mol. Biol. 151:389-409 (1981); Blake, R. D. & Hinds, P. W., J. Biomol. Struct. Dynam. 2:593-606 (1984); Hernan, R. A., et al., Biochemistry 31:8619-8627 (1992)]. Optimal polypeptide expression in E. coli, can be accomplished by the use of silent codon substitution mutagenesis to replace codons used more frequently for expression in mammalian cells with codons used more frequently for expression in E. coli.

Alternatively, in a different strategy, the protein-encoding region containing mammalian codons can be transformed into bacteria that are specifically designed to express commonly used eukaryotic codons bacterial that are rarely used in bacteria [Kane, J. Curr Opin Biotechnol.; 6:494-500 (1995)], for example, Rosetta strains expressing tRNA genes corresponding to the codons that are rarely used in E. coli.

An operon contains expression-regulating elements in addition to a group of closely linked genes that produce a single messenger RNA (mRNA) molecule in transcription. The expression-regulating elements include a promoter and an operator. Promoter sequences are generally found upstream of (that is 5′ to) the transcription start site; operator sequences may be located either upstream or downstream of the transcription start site. Promoters and operators used in the present invention are preferably functional in the bacterial environment.

A promoter is a DNA site to which RNA polymerase binds to initiate transcription of nearby genes. In one embodiment, the promoter is a naturally occurring promoter. For E. coli, promoter sequences have been extensively characterized [for a review, see Hawley, D. et al. Nucleic Acids Res. 11(8):2237-55 (1983)]. Alternatively, promoters used can be synthetic promoters. In particular, some synthetic promoters have been found to enhance transcription in a particular bacterial host cell. By way of illustration, the tac promoter is a hybrid of the E. coli trp promoter and the E. coli lac promoter, and directs transcription more efficiently than either lac or trp in E. coli cells [de Boer, H. et al. Proc Natl Acad Sci U S A 80(1):21-5 (1983)].

An operator is a DNA site to which a repressor protein binds to inhibit expression of nearby genes; operators serve as a switch turning transcription on and off in response to factors in the cellular environment. In one embodiment, the operator is a naturally occurring operator. An example of a naturally occurring operator in E. coli cells is the lac operator to which the Lac repressor binds. In the presence of an inducer of the lac operon, the Lac repressor releases from the operator, allowing transcription to proceed in an inducible fashion. In another embodiment, the operator is a synthetic operator. Effects of alteration of the lac operator sequences have been examined in E. coli [Stewart V., et al., J. Bacteriol. 185(7): 2104-2111 (2003)].

The operator may consist of a primary operator sequence, or may include auxiliary operator sequences in addition to the primary operator sequence. For example, the lac operon contains a primary operator sequence that is an inverted repeat in addition to two auxiliary operator sequences that provide maximum repression.

In the bacterial genome, operators can control expression of both structural genes necessary for survival of the bacteria and of regulatory genes, such as repressors. In the present invention, operators control expression of an Aurora enzyme and a phosphatase; in addition, the operator may control expression of one or more structural genes or regulatory genes.

The bacterially functional operon may contain components derived from bacterial sequences. In another embodiment, the bacterially functional operon contains components derived from bacteriophage sequences. A bacteriophage is a virus that infects bacteria. Bacteriophages belong to at least one of twelve distinct families. Generally, a given phage can infect only one species of bacteria or a few related species of bacteria. For example, coliphages from different families infecting E. coli include lambda, T4, and T7. Thus in another embodiment, the sequence of one or more components of the operon is derived from a phage that is capable of infecting the species of the particular bacterial host cell used. Bacteriophages also contain regulatory genes; examples are the lambda repressor and cro repressor responsible for directing the lysis/lysogeny decision of bacteriophage lambda.

The bacterially functional operon may also contain components derived from more than one species of bacteria, more than one species of phage, or combinations thereof. For example, a promoter from phage may be used in combination with a bacterial operator to direct expression of the Aurora encoding sequence and the phosphatase encoding sequence.

In another aspect, the invention is directed to a process comprising:
a) introducing into a bacterial host cell a polynucleotide comprising a sequence encoding an engineered Aurora enzyme;
b) growing the bacterial host cell under conditions whereby the engineered Aurora enzyme is expressed;
c) lysing the bacterial host cell, thereby producing a bacterial cell extract; and
d) separating the engineered Aurora enzyme from the bacterial cell extract.

In a yet another aspect, the invention is directed to a process comprising:
a) introducing into a bacterial host cell a polynucleotide comprising a first sequence encoding an engineered Aurora enzyme sequence and a second sequence encoding a phosphatase;
b) growing the bacterial host cell under conditions whereby the engineered Aurora enzyme and the phosphatase are co-expressed;
c) lysing the bacterial host cell, thereby producing a bacterial cell extract; and
d) separating the engineered Aurora enzyme from the bacterial cell extract.

In another embodiment of the process the first polynucleotide and the second polynucleotide reside on a single expression vector. In another embodiment of the process, the phosphatase is lambda phage phosphatase. In another embodiment of the process, the bacterial host cell is E. coli.

Introduction of single or multiple expression vectors into the bacterial host cells can be executed by techniques known in the art such as, for example, heat shock or electroporation. Growth conditions may vary depending on the nature of the bacterial strain, and the expression vectors used; optimal growth conditions can be readily ascertained by those skilled in the art. Many commercially available plasmids permit inducible expression of the desired protein products.

Isolation of the Aurora enzyme from the bacterial host cell can be performed by engineering the bacteria such that the Aurora enzyme is secreted from the bacterial host cell, followed by separation of the secreted Aurora enzyme from the bacterial host cell. In another embodiment, isolation of the Aurora enzyme from the bacterial host cell is performed by lysing the bacterial host cell thereby producing a bacterial cell extract, followed by separation of the Aurora enzyme from the bacterial cell extract.

Lysis of the bacterial host cells can be performed by use of well known techniques. Gentle techniques for cell disruption include, for example, freeze-thaw lysis, osmotic lysis, detergent lysis, and enzymatic lysis. More vigorous techniques for cell disruption include, for example, vortexing, sonication, microfluidization, pressing through a French pressure cell, and homogenization with glass beads. Other techniques include grinding with a mortar and pestle, and homogenizing with a mechanical device such as a blender or a Dounce grinder. The foregoing techniques may be used singly or in combination to disrupt cells. In general, cells are preferably kept chilled during the disruption procedure. The cell lysate typically includes, for example, cellular debris, nucleic acids such as RNA and DNA, proteins other than the Aurora enzyme, for example, chaperone proteins, and small molecules commonly present in the cellular milieu such as glutathione.

Like cell disruption, the separation of the Aurora enzyme from the bacterial cell extract can be accomplished by any of a number of techniques known in the art. Techniques that can be used to separate the Aurora enzyme from the cell lysate include precipitation, buffer exchange, preparative gel electrophoresis, chromatography, and the like.

For polypeptides of the invention comprising unnatural amino acids, methods of production such as, for example, nonsense suppression [Noren, C., et al., Science 244:182-188 (1989); Cload, S. T., et al., Chem. Biol. 3:1033-1038 (1996)] of a codon or codons in E. coli, may be used. Alternatively, in vitro protein biosynthesis methods [reviewed by Muir, T. W. in Annu. Rev. Biochem. 72:249-289 (2003)] may be employed.

Aurora Enzymatic Assays

The Aurora enzymes provided herein are useful for functional and structural studies of Aurora. Thus in another aspect, the invention is directed to a method of identifying modulators of Aurora enzymatic activity.

In another aspect, the invention is directed to a method of identifying modulators of Aurora activity, the method comprising:
a) combining in a first mixture an engineered Aurora polypeptide and a substrate with a compound and combining in a second mixture the polypeptide and the substrate without the compound;
b) placing the first mixture and the second mixture under a condition where the polypeptide is enzymatically active, and
c) determining a first extent of phosphorylation of the substrate in the first mixture and a second extent of phosphorylation of the substrate in the second mixture;
wherein a difference in the first extent of phosphorylation and the second extent of phosphorylation indicates that the compound is a modulator of Aurora activity.

In one embodiment of the method, the first extent of phosphorylation is less than the second extent of phosphorylation, and the compound is an Aurora inhibitor. Alternatively, in a different embodiment of the method, the first extent of phosphorylation is greater than the second extent of phosphorylation and the compound is an Aurora activator.

Substrates may be full length proteins, or the appropriate corresponding peptides containing the region phosphorylated by Aurora. Representative substrates of Aurora-B, for example, include but are not limited to topoisomerase II alpha, inner centromere protein (INCENP), survivin, borealin and histone H3.

The method can be performed using enzymatic assays that will be known to one skilled in the art. Such enzymatic assays include radiometric assays such as the scintillation proximity assay (SPA), luminescence assays, fluorescence-based assays using, for example, fluorescence intensity, fluorescence resonance energy transfer (FRET), or fluorescence polarization (FP) as a readout, and kinase assays using a different enzymatic activity (such as, for example, protease cleavage) as a readout.

The invention is further illustrated by the following non-limiting examples.

### EXAMPLES

The cloning of expression constructs for Aurora-A and Aurora-B enzymes is described in Example 1 and Example 2, respectively. As described in Example 1, a plasmid for coexpression of an Aurora enzyme and a phosphatase was prepared as follows. A parent plasmid that contained a phosphatase-encoding sequence was constructed, and subsequently the Aurora-encoding sequence was subcloned into the parent plasmid. The resulting plasmid contains the Aurora-encoding sequence and the phosphatase-encoding sequence each downstream of a respective Shine-Dalgarno sequence and under the control of a single promoter. The Aurora constructs are designed to express Aurora enzyme as a fusion protein with a solubilizing tag. As described in Example 2, Aurora-B constructs not coexpressing a phosphatase were also prepared.

Typical protocols for the expression, purification, and crystallization of Aurora-A protein are given in Example 3 along with autophosphorylation and activity assays. Example 4 provides a representative protocol for the expression and purification of Aurora-B protein. Example 5 describes an illustrative assay for Aurora-B enzymatic activity.

### Example 1

### Preparation of Aurora-A Expression Constructs

Preparation of Phosphatase Gene-Containing Plasmids for a Two-Gene Operon Expression of Kinases Toxic in E. Coli

The following describes the construction of a minioperon to coexpress phosphatase with toxic and/or multiply phosphorylated kinases in E. coli. The parent vector pGEX-6P-1 (GE Healthcare) contains the following: a lac operator, a tac promoter, an RBS sequence, a GST-encoding sequence, followed by a Prescission™ protease cleavage site encoding sequence, followed by a multiple cloning site. The vector allows cloning of cleavable GST fusion proteins. Downstream of the multiple cloning site are an ampicillin resistance gene, sequences necessary for plasmid replication and the lac repressor gene.

pGEX6P-1 plasmid was linearized with the XhoI restriction endonuclease, dephosphorylated with calf intestinal phosphatase, and gel-purified. Phosphatase genes (human protein phosphatase-1 [PP 1] α, β and γ (OriGene Technologies) as well as the λ-phage phosphatase gene [λPP] (New England BioLabs)) were amplified by Tgo polymerase (Roche Diagnostics). The 5′ primers used for amplification included an XhoI site as well as an intervening DNA sequence containing a Shine-Dalgarno ribosome-binding sequence [RBS] and designed to separate two genes of the two-gene minioperon. The 3′ primers used contained a SalI site. For human PP1α (GenBank ID: 45827796; locus: NM_{—}002708) SEQ ID NO:11 was used as the 5′ primer, and SEQ ID NO:12 was used as the 3′ primer.

SEQ ID NO:11

GATCACTCGAGCAATTTCACACAGGAAACAGTATTCATGTCCGACAGCGA
GAAGCTCAACCTGGAC

SEQ ID NO:12

CTGAGCACGTCGACTCATTTCTTGGCTTTGGCGGAATTGCGGGGT
GGGGTG

For human PP1β (GenBank ID: 46249374; locus: NM_{—}002709). SEQ ID NO:13 was used as the 5′ primer and SEQ ID NO:14 was used as the 3′ primer.

SEQ ID NO:13

GATCACTCGAGCAATTTCACACAGGAAACAGTATTCATGGCGGACGGGGA
GCTGAACGTGGACAGCC

SEQ ID NO:14

CTGAGCACGTCGACTCACCTTTTCTTCGGCGGATTAGCTGTTCGAGG

For human PP1γ (GenBank ID: 4506006; locus: NM_{—}002710) SEQ ID NO:15 was used as the 5′ primer and SEQ ID NO:16 was used as the 5′ primer.

SEQ ID NO:15

GATCACTCGAGCAATTTCACACAGGAAACAGTATTCATGGCGGATTTAGA
TAAACTCAACATCGACAGC

SEQ ID NO:16

CTGAGCACGTCGACTCATTTCTTTGCTTGCTTTGTGATCATACCCCTTGG

The λ phage phosphatase gene (GenBank ID: 215160; locus: AAA96594) was amplified from lambda DNA (New England BioLabs) with SEQ ID NO:17 as the forward (5′) primer (containing the XhoI cloning site and the Shine-Dalgarno [or ribosome-binding site (RBS) sequence]), and SEQ ID NO:18 as the reverse (3′) primer (containing a SalI site). Nucleotides 6-11 of SEQ ID NO:17 correspond to the XhoI cloning site, and nucleotides 12-36 of SEQ ID NO:17 correspond to the RBS sequence, which is followed by the start codon and the protein encoding sequence for the subsequent nine residues of λ phage phosphatase. Nucleotides 9-14 of SEQ ID NO:18 correspond to the SalI site.

SEQ ID NO:17

GATCACTCGAGCAATTTCACACAGGAAACAGTATTCATGCGCTATTACGA
AAAAATTGATGGCAGC

SEQ ID NO:18

CTGAGCACGTCGACTCATGCGCCTTCTCCCTGTACCTGAATCAATG

The PCR-amplified phosphatase fragments were gel-purified, cut with XhoI and SalI, and cloned into the XhoI site of pGEX-6P-1 using the Rapid DNA Ligation Kit (Roche Diagnostics Corporation) as recommended by the manufacturer. This step produced an intermediate construct with a phosphatase gene, the second gene of the two-gene operon, that could now be reopened with BamHI and XhoI restriction endonucleases for cloning of the Aurora DNA fragments.

The plasmids resulting from insertion of the λPP sequence with the correct orientation of the cloned insert, termed pGEX6P1-λPP, were used for the further cloning described herein.

Preparation of Mouse Aurora-A Expression Plasmids

Three mouse Aurora-A constructs were prepared: a longer construct used in enzymatic assays and two shorter constructs used in crystallography. For mouse Aurora-A, numbering refers to the full-length, wild type sequence of mouse Aurora-A isoform 1, provided here as SEQ ID NO:19. The mouse Aurora-A constructs also contain three humanizing and stabilizing mutations as described below.

| | |
|---|---|
| MDRCKENCVS RPVKTTVPFG PKRVLVTEQI PSQNLGSASS GQAQRVLCPS | SEQ ID NO:19 |
| NSQRVPSQAQ KLGAGQKPAP KQLPAASVPR PVSRLNNPQK NEQPAASGND | |
| SEKEQASLQK TEDTKKRQWT LEDFDIGRPL GKGKFGNVYL ARERQSKFIL | |
| ALKVLFKTQL EKANVEHQLR REVEIQSHLR HPNILRLYGY FHDATRVYLI | |
| LEYAPLGTVY RELQKLSKFD EQRTATYITE LANALSYCHS KRVIHRDIKP | |
| ENLLLGSNGE LKIADFGWSV HAPSSRRTTM CGTLDYLPPE MTEGRMHDEK | |
| VDLWSLGVLC YEFLVGMPPF EAHTYQETYR RISRVEFTFP DFVTEGARDL | |
| ISRLLKHNAS QRLTLAEVLE HPWIKANSSK PPTGHTSKEP TSKSS | |

The longer construct corresponds to residues 107-403 of the human sequence (i.e., mouse Aurora-A residues 98-395; 5′ primer SEQ ID NO:20; 3′ primer SEQ ID NO:21)

| | |
|---|---|
| CAGACGGATGGGGAAATGATTCTGAAAAGGAGC | SEQ ID NO:20 |
| CAGTCCTCGAGCTAAGATGATTTGCTGGTTGGCTC | SEQ ID NO:21 |

The first shorter construct encompasses residues corresponding to human residues 125-391 (i.e. mouse Aurora-A residues 116-381 with a serine added to the C-terminal end to mimic the end of the human protein). The DNA sequence encoding this portion of the enzyme was amplified from 15-day mouse embryonic cDNA (Ambion) as template DNA by Tgo polymerase with SEQ ID NO:22 as the 5′ primer, and SEQ ID NO:23 as the 3′ primer.

SEQ ID NO:22

CAGACGGATCCAAACGTCAGTGGACTTTGGAAGATTTTGACATTGGC

SEQ ID NO:23

CAGTCCTCGAGTCAGGACGGTTTGGAAGAATTAGCTTTGATCCAAGGG

The three humanizing and stabilizing mutations were introduced by site-directed mutagenesis using the QuikChange Site-Directed Mutagenesis Kit from Stratagene as recommended by the manufacturer (human numbers given in each pair): Asn173→Gly (mouse Aurora-A residue 164) (5′ primer: SEQ ID NO:24; 3′ primer: SEQ ID NO:25); Lys227→Arg (mouse-A residue 218) (5′ primer: SEQ ID NO:26; 3′ primer: SEQ ID NO:27); and Met289→Leu (mouse Aurora-A residue 280) (5′ primer: SEQ ID NO:28; 3′ primer: SEQ ID NO:29).

SEQ ID NO:24

CAGCTGGAGAAGGCGGGCGTGGAGCACCAGCTTCGGAG

SEQ ID NO:25

CTCCGAAGCTGGTGCTCCACGCCCGCCTTCTCCAGCTG

SEQ ID NO:26

GAGCTCCAAAAACTCTCCCGGTTTGACGAGCAGAGAACAGC

SEQ ID NO:27

GCTGTTCTCTGCTCGTCAAACCGGGAGAGTTTTTGGAGCTC

SEQ ID NO:28

CCAGGAGAACCACATTGTGTGGCACCCTGGACTACCTGCCC

SEQ ID NO:29

GGGCAGGTAGTCCAGGGTGCCACACAATGTGGTTCTCCTGG

The second shorter pGEX6P-1-based clone (clone A4-20-1-23-R5), contained mouse residues 117-395 (human residues 126-403) except that the C-terminal-most residues KPPTGHTSKEPTSKSS (SEQ ID NO:30) of mouse Aurora A were replaced with KPSNGQNKESASKQS (SEQ ID NO:31). SEQ ID NO:32 was used as the 3′ primer to create this clone.

SEQ ID NO:32

CAGTCCTCGAGTCAAGACTGTTTAGAAGCAGATTCTTTGTTCTGACCGTT
GGACGGTTTGGAAGAATTAGCTTTGATCCAAGGG

A Gly133→Val variant (human residue Gly142) was identified in the above clone (A4-20-1-23-R5). The Gly133→Val mutation in the A4-20-1-23-R5 clone was fixed by site-directed mutagenesis of the original altered gene cloned in a dual kinase-phosphatase expression plasmid as indicated above using SEQ ID NO:33 as the 5′ primer and SEQ ID NO:34 as the 3′ primer. The resulting reverted clone had the sequence provided as SEQ ID NO:35.

SEQ ID NO:33

GACATTGGCCGCCCACTAGGAAAAGGGAAGTTTGGAAATGTCTACTTGGC
GCGG

SEQ ID NO:36

CCGCGCCAAGTAGACATTTCCAAACTTCCCTTTTCCTAGTGGGCGGCCAA
TGTC

The Aurora portion of the sequences of the resulting mutated constructs are provided in SEQ ID NO:35, SEQ ID NO:36, and SEQ ID NO: 37.

| | |
|---|---|
| GNDSEKEQAS LQKTEDTKKR QWTLEDFDIG RPLGKGKFGN VYLARERQSK | SEQ ID NO:35 |
| FILALKVLFK TQLEKAGVEH QLRREVEIQS HLRHPNILRL YGYFHDATRV | |
| YLILEYAPLG TVYRELQKLS RFDEQRTATY ITELANALSY CHSKRVIHRD | |
| IKPENLLLGS NGELKIADFG WSVHAPSSRR TTLCGTLDYL PPEMIEGRMH | |
| DEKVDLWSLG VLCYEFLVGM PPFEAHTYQE TYRRISRVEF TFPDFVTEGA | |
| RDLISRLLKH NASQRLTLAE VLEHPWIKAN SSKPPTGHTS KEPTSKSS | |
| KRQWTLEDFD IGRPLGKGKF GNVYLARERQ SKFILALKVL FKTQLEKAGV | SEQ ID NO:36 |
| EHQLRREVEI QSHLRHPNIL RLYGYFHDAT RVYLILEYAP LGTVYRELQK | |
| LSRFDEQRTA TYITELANAL SYCHSKRVIH RDIKPENLLL GSNGELKIAD | |
| FGWSVHAPSS RRTTLCGTLD YLPPEMIEGR MHDEKVDLWS LGVLCYEFLV | |
| GMPPFEAHTY QETYRRISRV EFTFPDFVTE GARDLISRLL KHNASQRLTL | |
| AEVLEHPWIK ANSSKPS | |
| RQWTLEDFDI GRPLGKGKFG NVYLARERQS KFILALKVLF KTQLEKAGVE | SEQ ID NO:37 |
| HQLRREVEIQ SHLRHPNILR LYGYFHDATR VYLILEYAPL GTVYRELQKL | |
| SRFDEQRTAT YITELANALS YCHSKRVIHR DIKPENLLLG SMGELKIADF | |
| GWSVHAPSSR RTTLCGTLDY LPPEMTEGRM HDEKVDLWSL GVLCYEFLVG | |
| MPPFEAHTYQ ETYRRISRVE FTFPDFVTEG ARDLISRLLK HNASQRLTLA | |
| EVLEHPWIKA NSSKPSNGQN KESASKQS | |

The sequences of the constructs and expected molecular masses of the resulting proteins are provided in Table A.

TABLE A*

| | | Expected Molecular |
|---|---|---|
| Constuct description | Sequence | Mass |
| 98-395 mouse (corresponds | SEQ ID NO: 35 | 34,828.6 Da |
| to 107-403 human) | | |
| 116-381 mouse (corresponds | SEQ ID NO: 36 | 31,500.1 Da |
| to 125-391 human) | | |
| A4-20-1-23-R5 reverted | SEQ ID NO: 37 | 32,673.3 Da |

*Each construct contains an additional GPLGS (SEQ ID NO: N) at its N-terminus and the three mutations.

### Example 2

### Preparation of Mouse Aurora-B Expression Constructs

Mouse Aurora-B was amplified by Tgo polymerase with SEQ ID NO:38 as the 5′ primer, SEQ ID NO:39 as the 3′ primer, and 15-day mouse embryonic cDNA (BD Biosciences) as template DNA.

SEQ ID NO:38

GACACGGATCCAAACGTCAGTTCACTATTGACAACTTTGAGATTGGGCGT
CCTTTGGGCAAAGGC

SEQ ID NO:39

GACGACTCGAGTCAGGACGGCCTCCTTGAGTTGGCCCGGACCCAAGG
GTGAGC

The PCR-amplified fragments were gel-purified, cut with BamHI and XhoI, and cloned into the BamHI and XhoI sites of either the pGEX6P1-λPP plasmid or the pGEX6P1 plasmid which did not contain the λ phage phosphatase encoding sequence. The amplified fragment encodes mouse Aurora-B residues 74 (except that Lys-Gln-Pro of the mouse sequence have been replaced with human Lys-Arg-Gln) to 338 (plus a Pro-Ser added to the C-terminus of the sequence to mimic the end of the mouse Aurora-A crystallography construct). The mouse residues of Aurora-B correspond to residues 125-391 of human Aurora-A.

Surface-Residue Mutagenesis in Mouse Aurora-B

Wild-type full-length or truncated versions of mouse Aurora-B copurify from E. coli lysates in approximately 1:1 molar ratio with the bacterial chaperone protein GroEL. By analyzing the three-dimensional structure of humanized/stabilized mouse Aurora-A and assuming that the structure of mouse Aurora-B would be nearly identical to that of mouse Aurora-A, we initially identified 7 hydrophobic surface residues in mouse Aurora-B that correspond to polar residues in mouse and human Aurora-A. The following residues were mutated by site-directed mutagenesis using the QuikChange Site-Directed Mutagenesis Kit from Stratagene as recommended by the manufacturer, to stabilize the truncated Aurora-B in solution and reduce the amount of copurifying GroEL, thereby producing the mAurB 7X construct: Leu215→Ser (human Aurora-A residue: Ser266; mouse Aurora-A residue: Ser257; 5′ primer: SEQ ID NO:40; 3′ primer SEQ ID NO:41); Leu233→Ser (human Aurora-A residue: Ser284; mouse Aurora-A residue: Ser275; 5′ primer: SEQ ID NO:42; 3′ primer: SEQ ID NO:43); Met258→Lys (human Aurora-A residue: Lys309; mouse Aurora-A residue: Lys299; 5′ primer:SEQ ID NO:44; 3′ primer: SEQ ID NO:45); Val291→Ser (human Aurora-A residue: Ser342; mouse Aurora-A residue: Ser333; 5′ primer: SEQ ID NO:46; 3′ primer: SEQ ID NO:47); Pro302→Thr (human Aurora-A residue: Thr353; mouse Aurora-A residue: Thr344; 5′ primer: SEQ ID NO:48; 3′ primer: SEQ ID NO:49); Trp318→Ser (human Aurora-A residue: Ser369; mouse Aurora-A residue: Ser360); and Pro322→Thr (human Aurora-A residue: Met373; mouse Aurora-A residue: Thr364): for both Trp318→Ser and Pro322→Thr; 5′ primer: SEQ ID NO:50; 3′ primer: SEQ ID NO:51). The protein sequence of the mAurB 7X construct is provided as SEQ ID NO:52.

| | |
|---|---|
| AACCTGCTGTTAGGTTCCCAGGGAGAACTGAAG | SEQ ID NO:40 |
| CTTCAGTTCTCCCTGGGAACCTAACAGCAGGTT | SEQ ID NO:41 |
| GTGCATGCCCCATCCTCCAGGAGGAAGACCATGTGC | SEQ ID NO:42 |
| GCACATGGTCTTCCTCCTGGAGGATGGGGCATGCAC | SEQ ID NO:43 |
| CGCATGCATAATGAAAAAGTAGATCTATGGTGC | SEQ ID NO:44 |
| GCACCATAGATCTACTTTTTCATTATGCATGCG | SEQ ID NO:45 |
| GAGACGTATCGTCGGATTTCCAAGGTGGACCTGAAGTTC | SEQ ID NO:46 |
| GAACTTCAGGTCCACCTTGGAAATCCGACGATACGTCTC | SEQ ID NO:47 |
| TTCCCCTCTTCTGTGACCTCGGGCGCCCAGGAC | SEQ ID NO:48 |
| GTCCTGGGCGCCCGAGGTCACAGAAGAGGGGAA | SEQ ID NO:49 |
| CTCAAACATAACCCCTCCCAACGGCTGACCCTGGCGGAGGTTGCA | SEQ ID NO:50 |
| TGCAACCTCCGCCAGGGTCAGCCGTTGGGAGGGGTTATGTTTGAG | SEQ ID NO:51 |
| KRQFTIDNFE IGRPLGKGKF GNVYLAREKK SRFIVALKIL FKSQIEKEGV | SEQ ID NO:52 |
| EHQLRREIEI QAHLKHPNIL QLYNYFYDQQ RIYLILEYAP RGELYKELQK | |
| SRTFDEQRTA TIMEELSDAL TYCHKKKVIH RDIKPENLLL GSQGELKIAD | |
| FGWSVHAPSS RRKTMCGTLD YLPPEMIEGR MHNEKVDLWC IGVLCYELMV | |
| GNPPFESPSH SETYRRISKV DLKFPSSVTS GAQDLISKLL KHNPSQRLTL | |
| AEVAAHPWVR ANSRRPS | |

mAurB 7X was expressed in E. coli, purified as described below, and analyzed by electrospray mass spectrometry to verify the integrity of the purified protein. Approximately 50% of the protein was found to undergo proteolysis between Ser291 (mutated from Val291) and Lys292. A construct missing the Val291→Ser and Met258→Lys mutations (referred to as mAurB 5X) was purified, and analyzed by mass spec to determine the extent of proteolysis between Val291 and Lys292. Even in this wild type configuration, proteolysis still affected approximately 25% of total GST-tagged protein. Two additional conservative mutations were introduced to eliminate the major site of proteolysis: Val291→Leu and Lys292→Arg referred to as mAurB 8X VK→LR (5′ primer: SEQ ID NO:53; 3′ primer: SEQ ID NO:54) and Val291→Leu and Lys292→Gln referred to as mAurB 8X VK→LQ (5′ primer: SEQ ID NO:55; 3′ primer: SEQ ID NO:56). The protein sequences of the mAurB 8X VK→LR and mAurB 8X VK→LQ are provided in SEQ ID NO:57 and SEQ ID NO:58, respectively.

| | |
|---|---|
| CCACAGTGAGACGTATCGTCGGATTCTGCGCGTGGACCTGAAGTTCC | SEQ ID NO:53 |
| GGAACTTCAGGTCCACGCGCAGAATCCGACGATACGTCTCACTGTGG | SEQ ID NO:54 |
| GTGAGACGTATCGTCGGATTCTGCAGGTGGACCTGAAGTTCC | SEQ ID NO:55 |
| GGAACTTCAGGTCCACCTGCAGAATCCGACGATACGTCTCAC | SEQ ID NO:56 |
| KRQFTTDNFE IGRPLGKGKF GNVYLAREKK SRFIVALKIL FKSQIEKEGV | SEQ ID NO:57 |
| EHQLRREIEI QAHLKHPNIL QLYNYFYDQQ RIYLILEYAP RGELYKELQK | |
| SRTFDEQRTA TIMEELSDAL TYCHKKKVIH RDIKPENLLL GSQGELKIAD | |
| FGWSVHAPSS RRKTMCGTLD YLPPEMIEGR MHNEKVDLWC.IGVLCYELMV | |
| GNPPFESPSH SETYRRILRV DLKFPSSVTS GAQDLISKLL KHNPSQRLTL | |
| AEVAAHPWVR ANSRRPS | |
| KRQFTIDNFE IGRPLGKGKF GNVYLAREKK SRFIVALKIL FKSQIEKEGV | SEQ ID NO:58 |
| EHQLRREIEI QAHLKHPNIL QLYNYFYDQQ RIYLILEYAP RGELYKELQK | |
| SRTFDEQRTA TIMEELSDAL TYCHKKKVIH RDIKPENLLL GSQGELKIAD | |
| FGWSVHAPSS RRKTMCGTLD YLPPEMIEGR MHNEKVDLWC IGVLCYELMV | |
| GNPPFESPSH SETYRRILQV DLKFPSSVTS GAQDLISKLL KHNPSQRLTL | |
| AEVAAHPWVR ANSRRPS | |

Both mutant versions of the protein were no longer proteolyzed between Leu291 and Arg292 or Leu291 and Gln292, and both retained full enzyme activity.

### Example 3

### Aurora-A Expression, Purification, Activity Assays, and Crystallization

Expression and Purification

Expression plasmids were transformed into E. coli BL21(DE3) Star cells (Invitrogen), and grown with vigorous shaking at 37° C. in Terrific Broth supplemented with 200 μg/ml ampicillin. When the cultures reached an OD₆₀₀ of approximately 0.5-0.7, the temperature in the shaker was lowered to 17° C., and the cultures were incubated for 40 min prior to induction of protein expression with 0.2 mM IPTG and subsequent overnight incubation.

Cells were harvested 16-18 hrs post-induction by centrifugation and resuspension in a lysis buffer composed of 50 mM Tris-HCl pH 7.5, 1.0 M NaCl, 20 mM DTT supplemented with DNAse (Roche Diagnostics) and aprotinin (Sigma-Aldrich). Cells were lysed by passing the harvested culture four times through a microfluidizer (Microfluidics). Lysates were cleared by high-speed centrifugation, and loaded onto glutathione-Sepharose columns preequilibrated with 50 mM Tris-HCl pH 7.5 and 400 mM NaCl. Bound protein was eluted with 200 mM Tris-HCl pH 7.5, 500 mM NaCl, 15 mM reduced glutathione and 3 mM DTT. Following elution, the sample was treated with PreScission protease (rhinoviral 3C protease, GE Healthcare) to cleave off the GST tag, and simultaneously dialyzed against 50 mM Tris-HCl pH 7.5, 400 mM NaCl and 3 mM DTT at 4° C. to remove glutathione.

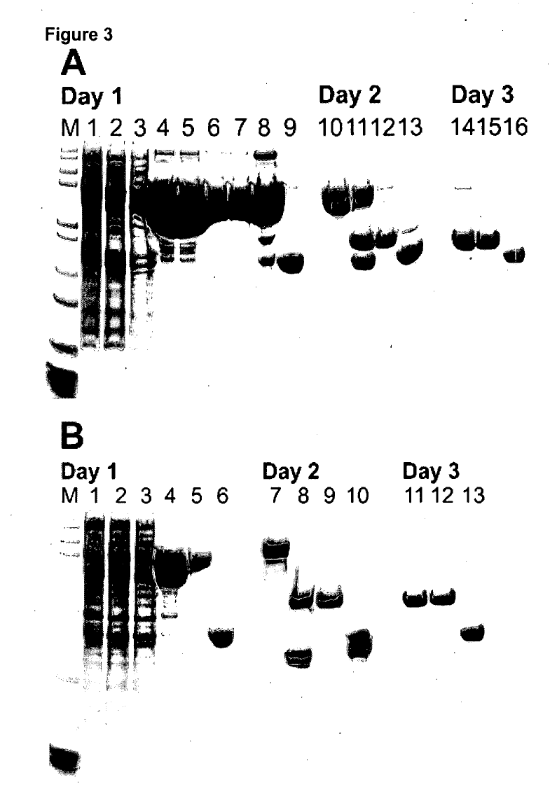

GST was eliminated by a second purification on the glutathione-Sepharose column. The protein sample was then diluted 1:1 with 25 mM Tris pH 7.5, and passed through another glutathione-Sepharose column connected in series to a Q-Sepharose column in order to remove small contaminating amounts of bacterial GST, GroEL, GroEL-Aurora-A complexes and nucleic acids. The purification steps are summarized in FIG. 3. For the protein in FIG. 3, panel A the protein recovery was 96% after concentration the Q Sepharose-subtracted protein from 0.4 mg/mL to 19.2 mg/mL and the total protein yield was 5.3 mg/L E. coli culture. For the protein in FIG. 3, panel B, the protein recovery was 67% after concentration the Q Sepharose-subtracted protein from 0.5 mg/mL to 8.9 mg/mL and the total protein yield was 6.2 mg/L E. coli culture.

Once purified, the samples were typically concentrated to approximately 6-8 mg/mL, centrifuged to remove the precipitate, aliquoted for crystallization experiments and activity assays, and snap-frozen in liquid nitrogen for storage at −80° C.

Autophosphorylation and Activity Assays

Protein samples were diluted to 1 mg/mL and incubated for 2 hrs at 4° C. with 1 mM ATP and 5 mM MgCl₂. Excess ATP was removed by extensive dialysis against 50 mM Tris-HCl pH 7.5, 200 mM NaCl, and 3 mM DTT.

Aurora A was titrated twofold in an assay buffer composed of 10 mM Tris-HCl pH 7.2, 10 mM MgCl₂, 0.1% BSA, 0.01% Triton X-100 and 1 mM DTT, and containing 120 nM of biotinylated histone H3 substrate peptide (Upstate Biotechnology). The kinase reactions were initiated by adding 10 μL ATP to a final volume of 60 μL and the final ATP concentration of 20 μM. The reactions were incubated at 25° C. for 60 min and terminated by adding EDTA to a final concentration of 200 mM.

To determine the Kₘ for ATP, ATP was titrated twofold in the assay buffer. The kinase reaction was initiated by adding 50 μL Aurora A (16 nM of insect cell-produced mouse enzyme with residues 98-395, 8 nM of the bacterially produced enzyme with residues 98-395, and 30 nM of the E. coli-expressed crystallography construct with residues 116-381) and histone H3 to a final volume of 60 μL and a final concentration of 120 nM.

To detect the assay product, the kinase reaction solution was combined with the detection buffer (the KF buffer) supplemented with 0.4 ng/μL phospho-histone H3 europium cryptate-conjugated antibody and 16 nM streptavidin-XL665 (both from Cisbio) in a 1:1 ratio in a 384-well plate. The mixture was incubated for 45 minutes at 25° C. and the plate was then scanned on the Analyst AD system. The fluorescence value F is defined as the ratio of fluorescent emissions at 620 and 665 nm. ΔF was generated by subtracting the average F value for negative control for enzyme activity from the F value in each reaction well. ΔF was plotted against enzyme concentration (for enzyme titrations) or against ATP concentration (for ATP titrations) with GraphPad Prism. The Kₘ values and confidence intervals were determined by fitting the ATP titration curve in GraphPad Prism to the following equation: y=(Vₘₐₓ*x)/(Kₘ+x), where y is ΔF/hr and x is an ATP concentration.

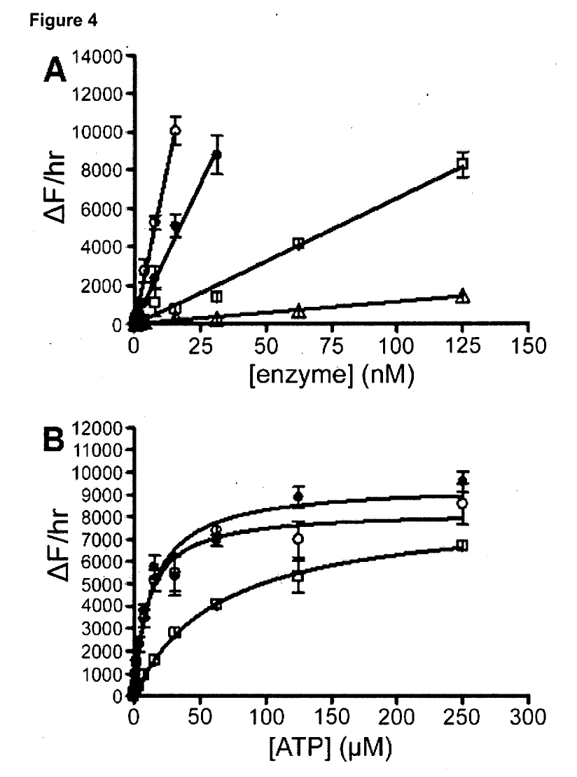

The shorter crystallography construct covering mouse residues 116-381 (human residues 125-391) was less active and showed a substantial increase in the Kₘ for ATP (FIG. 4) indicating that the extra N- and C-terminal residues may stabilize the ATP-bound form of the enzyme. The Gly142→Val substitution resulted in a strong decrease in enzyme activity when tested with the H3 peptide as the substrate (FIG. 4). This residue is located on the P-loop and is highly conserved among many protein kinases which suggests a key role it plays in kinase function.

Crystallization Conditions for Mouse Aurora-A

Crystals of the phosphorylated A4-20-1-23-R5 clone with small molecule inhibitors were grown by hanging-drop vapor diffusion at 20° C. in (1) 0.1 M Tris-HCl pH 7.0-7.5, 0.08-0.2 M ammonium sulfate and 30% PEG 3350; (2) 0.1 M PIPES pH 6.0, 0.1-0.2 M ammonium or lithium sulfate, and 25-30% PEG 3350; or (3) 0.1 M PIPES ph 6.0, 0.2 M ammonium sulfate, and 25% PEG 4000, 6000 or 8000.

Crystals of unphosphorylated mouse Aurora A 116-381 in complex with inhibitors were obtained by hanging-drop vapor diffusion at 20° C. against a reservoir of (1) 0.1 M bis-Tris propane pH 7.0, 1.8 M sodium acetate pH 7.0; (2) 1.0 M sodium/potassium phosphate pH 6.9; (3) 0.1 M bis-Tris propane pH 7.0, 2.0 M sodium chloride; (4) 0.1 M bis-Tris propane pH 7.0, 0.8 M lithium sulfate; (5) 0.1 M bis-Tris propane pH 7.0, 0.5 M potassium thiocyanate; (6) 0.1 M bis-Tris propane pH 7.0, 35% Tacsimate; plus additional conditions from the SaltRx, sodium malonate as well as PEG/Ion grid screens from Hampton Research.

All crystals for data collection were cryoprotected in mother liquors supplemented with 20% (v/v) glycerol or 20% (v/v) ethylene glycol for 0.5-1.5 min and immersion in liquid nitrogen.

Diffraction data were collected under standard cryogenic conditions on a Rigaku RU-3R rotating anode generator and an RAXIS-IV detector, processed and scaled with CrystalClear from Rigaku/Molecular Structure Corporation.

### Example 4

### Aurora-B Expression and Purification

Aurora-B protein was expressed in BL21(DE3) Star cells (Invitrogen). Cells were grown at 37° C. until OD₆₀₀=0.6-0.9. Temperature was then lowered to 18° C., cells were shaken for an additional 40 minutes. Protein expression was induced with 0.3 mM IPTG, and the cells were incubated with vigorous shaking overnight. Following the incubation, cells were harvested into a buffer composed of 50 mM Tris pH 7.5, 600 mM NaCl, and 20 mM DTT supplemented with endopeptidase inhibitors. Cells were lysed, insoluble matter was removed by high-speed centrifugation, and the cleared lysate was loaded onto a glutathione-Sepharose column preequilibrated with 50 mM Tris pH 7.5 and 400 mM NaCl. The column was washed thoroughly with the equilibration buffer, and the protein eluted with 100 mM Tris pH 7.5, 500 mM NaCl, 2 mM DTT and 10-15 mM reduced glutathione. Protein samples were dialyzed against two 4-L volumes of 50 mM Tris pH 7.5, 400 mM NaCl, and 2 mM DTT to remove glutathione. GST-tagged mouse Aurora-B was left uncleaved since mouse Aurora-B is unstable in the absence of a solubilizing tag and precipitates of solution when cleaved. At this step the protein was better than 95% pure. A 2-4 mL volume of uncleaved GST-mouse Aurora-B from the top of the eluted peak was frozen directly in the elution buffer in small aliquots at −80° C. to prevent loss of activity that occurs during extended dialysis at 4° C.

### Example 5

### Aurora-B Enzyme Activity Assay

Aurora-B enzyme preparations were tested for kinase activity in the presence and absence of compounds using a homogeneous time resolved fluorescence (HTRF) assay. In brief, the assay was performed by adding enzyme under appropriate reaction conditions to a tagged substrate. After the kinase reaction proceeds, quenching reagent is added, along with a first molecule consisting of a fluorescence donor conjugated to an antibody recognizing the phosphorylated substrate, and a second molecule that recognizes the tag and that is conjugated to a fluorescence acceptor. The extent of the kinase reaction is measured by time-resolved fluorescence transfer from the fluorescence donor to the fluorescence acceptor in a detection buffer, which occurs when the donor and acceptor are in proximity to one another on the phosphorylated substrate.

The assay was carried out as follows. The compound to be tested is added in a 1.5 μL volume to a multiwell plate. Substrate and enzyme are added to the plate so that enzyme reactions were carried out under final concentrations of 120 mM biotin-conjugated histone-H3 peptide substrate (Upstate Cat. No. 12-403), 7.5 nM in house Aurora-B, 300 μM ATP, 1 mM DTT, and 1X IMAP reaction buffer (Molecular Devices Cat. No. R7209) in a 61.5 μL volume. As a positive control, a 50:50 mixture of phosphorylated and unphosphorylated biotinylated histone H3 was used at a final total concentration of 120 nM histone H3. Reactions were incubated for 60 min (room temperature, 22° C.), and quenched by addition of 40 μL of 50 mM EDTA (to a final concentration of 20 mM).

A 5 μL aliquot of the quenched reaction was transferred to a separate plate containing 5 μL detection buffer (final concentrations: 125 ng/mL europium cryptate-conjugated α-phospho H3 antibody, 8 nM streptavidin-tagged XL665, 25 mM HEPES buffer (pH 7.0), 0.25 M KF, and 0.05% BSA). The detection solution was allowed to incubate at room temperature for 45 min, after which the detection reactions were read on an Analyst AD (LJL Biosystems). Excitation was for 400 μs in the 330-370 nm range. Emission was detected at 665 nm and 620 nm, corresponding to excited XL665 and unbound europium cryptate emissions respectively. Delta F is a value calculated from the ratio between the 665 nm and 620 nm emissions. The “ratio” is the counts per second (cps) at 665 nm divided by the cps at 620 nm multiplied by 10⁴. The “delta F” is the difference between the ratios for the sample well and the negative control.

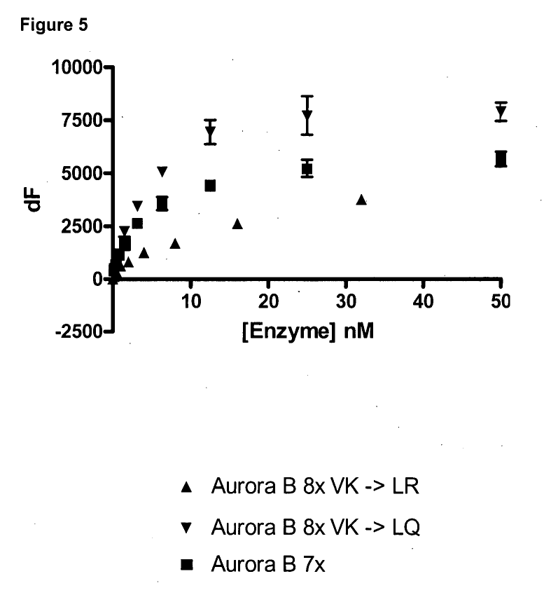

FIG. 5 shows kinase activity as a function of enzyme concentration for the Aurora B 8X.VK->LQ construct, the Aurora B 7X construct, and the Aurora B 8X.VK->LR construct. As shown in FIG. 5, each of the constructs shows measurable activity in the 1-25 nM range. Furthermore, a comparison of the activity of the Aurora B 7X construct at an enzyme concentration of 20 nM was made with commercially available Aurora-B at an enzyme concentration of 300 nM. As a positive control, 60 nM phospho-H3 was used in the absence of enzyme. The activity of the Aurora B 7X construct compares favorably with the activity of commercially available Aurora-B enzyme.

We claim:

1. A polypeptide comprising Aurora-B enzymatic activity, which polypeptide comprises a substitution mutation at a position corresponding to V286 of SEQ ID NO:7.

2. The polypeptide of claim 1 further comprising a substitution mutation at a position corresponding to K287 of SEQ ID NO:7.

3. The polypeptide of claim 1, wherein the substitution mutation is to an amino acid isosteric with Leu.

4. The polypeptide of claim 3 wherein the substitution mutation is to Ser, Leu, Ile, Thr, or Ala.

5. The polypeptide of claim 4 wherein the substitution mutation is to Ser or Leu.

6. The polypeptide of claim 1 wherein the polypeptide further comprises at least one substitution mutation at a position corresponding to residue L210 of SEQ ID NO:7 or L228 of SEQ ID NO:7.

7. The polypeptide of claim 6, wherein the polypeptide comprises the substitution mutation at the position corresponding to residue L210 of SEQ ID NO:7 and the substitution mutation at the position corresponding to residue L228 of SEQ ID NO:7.

8. The polypeptide of claim 7, wherein the substitution mutation at the position corresponding to residue L210 of SEQ ID NO:7 and the substitution mutation at the position corresponding to L228 of SEQ ID NO:7 are each independently to Ser or Thr.

9. The polypeptide of claim 7, wherein the polypeptide further comprises at least one substitution mutation in a position corresponding to residue P297 of SEQ ID NO:7 or P317 of SEQ ID NO:7.

10. The polypeptide of claim 1, wherein the polypeptide further comprises at least one substitution mutation in a position corresponding to residue P297 of SEQ ID NO:7 or P317 of SEQ ID NO:7.

11. The polypeptide of claim 10, wherein the polypeptide further comprises the substitution mutation at the position corresponding to residue P297 of SEQ ID NO:7 and the substitution mutation at the position corresponding to residue P317 of SEQ ID NO:7.

12. The polypeptide of claim 11 wherein the substitution mutation in the position corresponding to residue P297 of SEQ ID NO:7 and the substitution mutation in the position corresponding to residue P317 of SEQ ID NO:7 are each independently to Ser or Thr.

13. The polypeptide of claim 1 having a sequence derived from a mammalian Aurora-B.

14. The polypeptide of claim 13, wherein the sequence is derived from a human Aurora-B.

15. The polypeptide of claim 1, wherein the polypeptide further comprises a solubilizing tag.

16. A polynucleotide encoding the polypeptide of claim 1.

17. A vector comprising the polynucleotide of claim 16.

18. A bacterial host cell comprising the vector of claim 17.

19. The bacterial host cell of claim 18, wherein the bacterial host cell is E. coli.

20. A method of identifying modulators of Aurora activity, the method comprising:
a) combining in a first mixture the polypeptide of claim 1 and a substrate with a compound and combining in a second mixture the polypeptide and the substrate without the compound;
b) placing the first mixture and the second mixture under a condition where the polypeptide is enzymatically active, and
c) determining a first extent of phosphorylation of the substrate in the first mixture and a second extent of phosphorylation of the substrate in the second mixture;
wherein a difference in the first extent of phosphorylation and the second extent of phosphorylation indicates that the compound is a modulator of Aurora activity.

21. A polypeptide comprising a sequence selected from the group consisting of SEQ ID NO: 35, SEQ ID NO:36, and SEQ ID NO:37.

22. A polynucleotide encoding a polypeptide comprising a sequence selected from the group consisting of SEQ ID NO:35, SEQ ID NO:36, and SEQ ID NO:37.

23. A vector comprising the polynucleotide of claim 22.


SEQUENCE LISTING (58 sequences)

SEQ ID NO: 1 - protein, 265 aa, Mus musculus
NFEIGRPLGKGKFGNVYLAREKKSRFIVALKILFKSQIEKEGVEHQLRREIEIQAHLHHPNILRLYNYFYDRRRIYLILEYAPRGELYKELQKSRTFDEQRTATIMEELSDALTYCHKKKVIHRDIKPENLLLGLQGELKIADFGWSVHAPSLRRKTMCGTLDYLPPEMIEGRMHNEMVDLWCIGVLCYELMVGNPPFESPSHSETYRRIVKVDLKFPSSVPSGAQDLISKLLKHNPWQRLPLAEVAAHPWVRANSRRVLPPSAL

SEQ ID NO: 2 - protein, 265 aa, Homo sapiens
DFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPTGAQDLISKLLRHNPSERLPLAQVSAHPWVRANSRRVLPPSAL

SEQ ID NO: 3 - protein, 252 aa, Homo sapiens
DFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEAHTYQETYRRISRVEFTFPDFVTEGARDLISRLLKHNASQRPMLREVLEHPWI

SEQ ID NO: 4 - protein, 252 aa, Mus musculus
DFDIGRPLGKGKFGNVYLARERQSKFILALKVLFKTQLEKANVEHQLRREVEIQSHLRHPNILRLYGYFHDATRVYLILEYAPLGTVYRELQKLSKFDEQRTATYITELANALSYCHSKRVIHRDIKPENLLLGSNGELKIADFGWSVHAPSSRRTTMCGTLDYLPPEMIEGRMHDEKVDLWSLGVLCYEFLVGMPPFEAHTYQETYRRISRVEFTFPDFVTEGARDLISRLLKHNASQRLTLAEVLEHPWI

SEQ ID NO: 5 - protein, 265 aa, Rattus norvegicus
NFEIGRPLGKGKFGNVYLAREKKSRFIVALKILFKSQIEKEGVEHQLRREIEIQAHLHHPNILRLYNYFYDRRRIYLILEYAPRGELYKELQKSGTFDEQRTATIMEELSDALMYCHKKKVIHRDIKPENLLLGLQGELKIADFGWSVHAPSLRRKTMCGTLDYLPPEMIEGRMHNEMVDLWCIGVLCYELMVGNPPFESPSHSETYRRIVKVDLKFPSSMPLGAKDLISKLLKHNPSQRLPLEQVSAHPWVRANSRRVLPPSAL

SEQ ID NO: 6 - protein, 156 aa, Sus scrofa
IYLILEYAPRGELYKELQKCRTFDEQRTATIMEELADALIYCHGKKVIHRDIKPENLLLGLQGELKIADFGWSVHAPSLRRKTMRGTLDYLPPEMIEGRTHNEKVDLWCIGVLCYELLVGNPPFESASHNETYRRIVKVDLKFPPSVPAGAQDLIS

SEQ ID NO: 7 - protein, 344 aa, Homo sapiens
MAQKENSYPWPYGRQTAPSGLSTLPQRVLRKEPVTPSALVLMSRSNVQPTAAPGQKVMENSSGTPDILTRHFTIDDFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPTGAQDLISKLLRHNPSERLPLAQVSAHPWVRANSRRVLPPSALQSVA

SEQ ID NO: 8 - protein, 267 aa, Artificial Sequence
  Primer
KRQFTIDNFEIGRPLGKGKFGNVYLAREKKSRFIVALKILFKSQIEKEGVEHQLRREIEIQAHLKHPNILQLYNYFYDQQRIYLILEYAPRGELYKELQKSRTFDEQRTATIMEELSDALTYCHKKKVIHRDIKPENLLLGSQGELKIADFGWSVHAPSSRRKTMCGTLDYLPPEMIEGRMHNEKVDLWCIGVLCYELMVGNPPFESPSHSETYRRISKVDLKFPSSVTSGAQDLISKLLKHNPSQRLTLAEVAAHPWVRANSRRPS

SEQ ID NO: 9 - protein, 267 aa, Artificial Sequence
  Primer
KRQFTIDNFEIGRPLGKGKFGNVYLAREKKSRFIVALKILFKSQIEKEGVEHQLRREIEIQAHLKHPNILQLYNYFYDQQRIYLILEYAPRGELYKELQKSRTFDEQRTATIMEELSDALTYCHKKKVIHRDIKPENLLLGSQGELKIADFGWSVHAPSSRRKTMCGTLDYLPPEMIEGRMHNEKVDLWCIGVLCYELMVGNPPFESPSHSETYRRILRVDLKFPSSVTSGAQDLISKLLKHNPSQRLTLAEVAAHPWVRANSRRPS

SEQ ID NO: 10 - protein, 267 aa, Artificial Sequence
  Primer
KRQFTIDNFEIGRPLGKGKFGNVYLAREKKSRFIVALKILFKSQIEKEGVEHQLRREIEIQAHLKHPNILQLYNYFYDQQRIYLILEYAPRGELYKELQKSRTFDEQRTATIMEELSDALTYCHKKKVIHRDIKPENLLLGSQGELKIADFGWSVHAPSSRRKTMCGTLDYLPPEMIEGRMHNEKVDLWCIGVLCYELMVGNPPFESPSHSETYRRILQVDLKFPSSVTSGAQDLISKLLKHNPSQRLTLAEVAAHPWVRANSRRPS

SEQ ID NO: 11 - DNA, 66 bp, Artificial Sequence
  Primer
gatcactcgagcaatttcacacaggaaacagtattcatgtccgacagcgagaagctcaacctggac

SEQ ID NO: 12 - DNA, 51 bp, Artificial Sequence
  Primer
ctgagcacgtcgactcatttcttggctttggcggaattgcggggtggggtg

SEQ ID NO: 13 - DNA, 67 bp, Artificial Sequence
  Primer
gatcactcgagcaatttcacacaggaaacagtattcatggcggacggggagctgaacgtggacagcc

SEQ ID NO: 14 - DNA, 47 bp, Artificial Sequence
  Primer
ctgagcacgtcgactcaccttttcttcggcggattagctgttcgagg

SEQ ID NO: 15 - DNA, 69 bp, Artificial Sequence
  Primer
gatcactcgagcaatttcacacaggaaacagtattcatggcggatttagataaactcaacatcgacagc

SEQ ID NO: 16 - DNA, 50 bp, Artificial Sequence
  Primer
ctgagcacgtcgactcatttctttgcttgctttgtgatcataccccttgg

SEQ ID NO: 17 - DNA, 66 bp, Artificial Sequence
  Primer
gatcactcgagcaatttcacacaggaaacagtattcatgcgctattacgaaaaaattgatggcagc

SEQ ID NO: 18 - DNA, 46 bp, Artificial Sequence
  Primer
ctgagcacgtcgactcatgcgccttctccctgtacctgaatcaatg

SEQ ID NO: 19 - protein, 395 aa, Mus musculus
MDRCKENCVSRPVKTTVPFGPKRVLVTEQIPSQNLGSASSGQAQRVLCPSNSQRVPSQAQKLGAGQKPAPKQLPAASVPRPVSRLNNPQKNEQPAASGNDSEKEQASLQKTEDTKKRQWTLEDFDIGRPLGKGKFGNVYLARERQSKFILALKVLFKTQLEKANVEHQLRREVEIQSHLRHPNILRLYGYFHDATRVYLILEYAPLGTVYRELQKLSKFDEQRTATYITELANALSYCHSKRVIHRDIKPENLLLGSNGELKIADFGWSVHAPSSRRTTMCGTLDYLPPEMIEGRMHDEKVDLWSLGVLCYEFLVGMPPFEAHTYQETYRRISRVEFTFPDFVTEGARDLISRLLKHNASQRLTLAEVLEHPWIKANSSKPPTGHTSKEPTSKSS

SEQ ID NO: 20 - DNA, 33 bp, Artificial Sequence
  Primer
cagacggatggggaaatgattctgaaaaggagc

SEQ ID NO: 21 - DNA, 35 bp, Artificial Sequence
  Primer
cagtcctcgagctaagatgatttgctggttggctc

SEQ ID NO: 22 - DNA, 47 bp, Artificial Sequence
  Primer
cagacggatccaaacgtcagtggactttggaagattttgacattggc

SEQ ID NO: 23 - DNA, 48 bp, Artificial Sequence
  Primer
cagtcctcgagtcaggacggtttggaagaattagctttgatccaaggg

SEQ ID NO: 24 - DNA, 38 bp, Artificial Sequence
  Primer
cagctggagaaggcgggcgtggagcaccagcttcggag

SEQ ID NO: 25 - DNA, 38 bp, Artificial Sequence
  Primer
ctccgaagctggtgctccacgcccgccttctccagctg

SEQ ID NO: 26 - DNA, 41 bp, Artificial Sequence
  Primer
gagctccaaaaactctcccggtttgacgagcagagaacagc

SEQ ID NO: 27 - DNA, 41 bp, Artificial Sequence
  Primer
gctgttctctgctcgtcaaaccgggagagtttttggagctc

SEQ ID NO: 28 - DNA, 41 bp, Artificial Sequence
  Primer
ccaggagaaccacattgtgtggcaccctggactacctgccc

SEQ ID NO: 29 - DNA, 41 bp, Artificial Sequence
  Primer
gggcaggtagtccagggtgccacacaatgtggttctcctgg

SEQ ID NO: 30 - protein, 16 aa, Artificial Sequence
  Primer
KPPTGHTSKEPTSKSS

SEQ ID NO: 31 - protein, 15 aa, Artificial Sequence
  Primer
KPSNGQNKESASKQS

SEQ ID NO: 32 - DNA, 84 bp, Artificial Sequence
  Primer
cagtcctcgagtcaagactgtttagaagcagattctttgttctgaccgttggacggtttggaagaattagctttgatccaaggg

SEQ ID NO: 33 - DNA, 54 bp, Artificial Sequence
  Primer
gacattggccgcccactaggaaaagggaagtttggaaatgtctacttggcgcgg

SEQ ID NO: 34 - DNA, 54 bp, Artificial Sequence
  Primer
ccgcgccaagtagacatttccaaacttcccttttcctagtgggcggccaatgtc

SEQ ID NO: 35 - protein, 298 aa, Artificial Sequence
  Mutated Constructs
GNDSEKEQASLQKTEDTKKRQWTLEDFDIGRPLGKGKFGNVYLARERQSKFILALKVLFKTQLEKAGVEHQLRREVEIQSHLRHPNILRLYGYFHDATRVYLILEYAPLGTVYRELQKLSRFDEQRTATYITELANALSYCHSKRVIHRDIKPENLLLGSNGELKIADFGWSVHAPSSRRTTLCGTLDYLPPEMIEGRMHDEKVDLWSLGVLCYEFLVGMPPFEAHTYQETYRRISRVEFTFPDFVTEGARDLISRLLKHNASQRLTLAEVLEHPWIKANSSKPPTGHTSKEPTSKSS

SEQ ID NO: 36 - protein, 267 aa, Artificial Sequence
  Mutated Constructs
KRQWTLEDFDIGRPLGKGKFGNVYLARERQSKFILALKVLFKTQLEKAGVEHQLRREVEIQSHLRHPNILRLYGYFHDATRVYLILEYAPLGTVYRELQKLSRFDEQRTATYITELANALSYCHSKRVIHRDIKPENLLLGSNGELKIADFGWSVHAPSSRRTTLCGTLDYLPPEMIEGRMHDEKVDLWSLGVLCYEFLVGMPPFEAHTYQETYRRISRVEFTFPDFVTEGARDLISRLLKHNASQRLTLAEVLEHPWIKANSSKPS

SEQ ID NO: 37 - protein, 278 aa, Artificial Sequence
  Mutated Constructs
RQWTLEDFDIGRPLGKGKFGNVYLARERQSKFILALKVLFKTQLEKAGVEHQLRREVEIQSHLRHPNILRLYGYFHDATRVYLILEYAPLGTVYRELQKLSRFDEQRTATYITELANALSYCHSKRVIHRDIKPENLLLGSNGELKIADFGWSVHAPSSRRTTLCGTLDYLPPEMIEGRMHDEKVDLWSLGVLCYEFLVGMPPFEAHTYQETYRRISRVEFTFPDFVTEGARDLISRLLKHNASQRLTLAEVLEHPWIKANSSKPSNGQNKESASKQS

SEQ ID NO: 38 - DNA, 65 bp, Artificial Sequence
  Primer
gacacggatccaaacgtcagttcactattgacaactttgagattgggcgtcctttgggcaaaggc

SEQ ID NO: 39 - DNA, 53 bp, Artificial Sequence
  Primer
gacgactcgagtcaggacggcctccttgagttggcccggacccaagggtgagc

SEQ ID NO: 40 - DNA, 33 bp, Artificial Sequence
  Primer
aacctgctgttaggttcccagggagaactgaag

SEQ ID NO: 41 - DNA, 33 bp, Artificial Sequence
  Primer
cttcagttctccctgggaacctaacagcaggtt

SEQ ID NO: 42 - DNA, 36 bp, Artificial Sequence
  Primer
gtgcatgccccatcctccaggaggaagaccatgtgc

SEQ ID NO: 43 - DNA, 36 bp, Artificial Sequence
  Primer
gcacatggtcttcctcctggaggatggggcatgcac

SEQ ID NO: 44 - DNA, 33 bp, Artificial Sequence
  Primer
cgcatgcataatgaaaaagtagatctatggtgc

SEQ ID NO: 45 - DNA, 33 bp, Artificial Sequence
  Primer
gcaccatagatctactttttcattatgcatgcg

SEQ ID NO: 46 - DNA, 39 bp, Artificial Sequence
  Primer
gagacgtatcgtcggatttccaaggtggacctgaagttc

SEQ ID NO: 47 - DNA, 39 bp, Artificial Sequence
  Primer
gaacttcaggtccaccttggaaatccgacgatacgtctc

SEQ ID NO: 48 - DNA, 33 bp, Artificial Sequence
  Primer
ttcccctcttctgtgacctcgggcgcccaggac

SEQ ID NO: 49 - DNA, 33 bp, Artificial Sequence
  Primer
gtcctgggcgcccgaggtcacagaagaggggaa

SEQ ID NO: 50 - DNA, 45 bp, Artificial Sequence
  Primer
ctcaaacataacccctcccaacggctgaccctggcggaggttgca

SEQ ID NO: 51 - DNA, 45 bp, Artificial Sequence
  Primer
tgcaacctccgccagggtcagccgttgggaggggttatgtttgag

SEQ ID NO: 52 - protein, 267 aa, Mus musculus; identical to SEQ ID NO: 8

SEQ ID NO: 53 - DNA, 47 bp, Artificial Sequence
  Primer
ccacagtgagacgtatcgtcggattctgcgcgtggacctgaagttcc

SEQ ID NO: 54 - DNA, 47 bp, Artificial Sequence
  Primer
ggaacttcaggtccacgcgcagaatccgacgatacgtctcactgtgg

SEQ ID NO: 55 - DNA, 42 bp, Artificial Sequence
  Primer
gtgagacgtatcgtcggattctgcaggtggacctgaagttcc

SEQ ID NO: 56 - DNA, 42 bp, Artificial Sequence
  Primer
ggaacttcaggtccacctgcagaatccgacgatacgtctcac

SEQ ID NO: 57 - protein, 267 aa, Mus musculus; identical to SEQ ID NO: 9

SEQ ID NO: 58 - protein, 267 aa, Mus musculus; identical to SEQ ID NO: 10